(19) United States
(12) Patent Application Publication
(10) Pub. No.: US 2026/0013951 A1
(43) Pub. Date: Jan. 15, 2026

(54) SYSTEMS AND METHODS FOR WIRELESS LOCALIZATION INTEGRATION

(71) Applicant: Elucent Medical, Inc., Eden Prairie, MN (US)

(72) Inventors: Jason Hiltner, Eden Prairie, MN (US); Adam Fisher, Eden Prairie, MN (US); Bryan Dean, Eden Prairie, MN (US)

(21) Appl. No.: 19/332,584

(22) Filed: Sep. 18, 2025

Related U.S. Application Data
(63) Continuation of application No. 17/746,105, filed on May 17, 2022.
(60) Provisional application No. 63/189,394, filed on May 17, 2021.

(51) Int. Cl. A61B 34/20; A61B 34/30; A61B 90/00

(52) U.S. Cl. CPC A61B 34/20; A61B 34/30; A61B 90/361; A61B 2034/2051; A61B 2034/2072

(57) ABSTRACT

A wireless localization system including a pad with an exciter coil and a sensor coil, a tool or surgical robot including a wireless tag configured to generate a signal in response to a magnetic field generated by the exciter coil. The signal is detected by the sensor coil and a processor configured to determine the location of the tool.

### CROSS REFERENCE TO RELATED APPLICATIONS

This application is a continuation of U.S. patent application Ser. No. 17/746,105, filed on May 17, 2022, which claims priority to U.S. Provisional Patent Application No. 63/189,394, filed on May 17, 2021, the entire contents of which are incorporated herein by reference.

### TECHNICAL FIELD

The present disclosure relates systems, devices, assemblies, and methods for integrating wirelessly located marker tags into surgical and medical procedures. The systems, devices, assemblies, and methods find use in a variety of applications including integration with a surgical robot assembly.

### BACKGROUND

A common and serious challenge for many medical procedures is the accurate location of treatment areas. For example, the location of lesions, such as tumors that are to undergo treatment, including surgical resection, continues to present a challenge to the medical community. Existing systems are expensive, complex, time-consuming, and often unpleased for the patient.

Such issues are illustrated by the conventional surgical treatment of pulmonary nodules. In some cases where pulmonary nodules may be difficult to locate at conventional open surgery or at thoracoscopy, a hook wire, injection or visible dye, or a radionuclide is place in or around the nodule in an attempt to improve localization prior to removal. This procedure usually takes place in the computerized tomography (CT) suit prior to the removal of the nodule. The patient is then transported to the surgical unit and the surgeon cuts down on the wire, uses a radionuclide detector, or uses visual landmarks to localize and remove the nodule.

A similar type of procedure is done to localize pulmonary nodules prior to resection. In some cases where pulmonary nodules may be difficult to locate at conventional open surgery or at thoracoscopy, a hook wire, injection of visible dye, or a radionuclide is place in or around the nodule in an attempt to improve localization prior to removal. This procedure usually takes place in the CT suite prior to the removal of the nodule. The patient is then transported to the surgical unit and the surgeon cuts down on the wire, uses a radionuclide detector, or uses visual landmarks to localize and remove the nodule.

In addition, the tools used during a medical procedure are also difficult to locate. For example, the location of a hand-held tool utilized by a surgeon may not be known, other than intuitively by the surgeon. Any wired location sensor adds to the number of wires, tubes, etc. extending off from the hand-held tool-thereby reducing maneuverability of the tool.

Many other medical devices and procedures could benefit from improved tissue and tool localization. These include any procedure or test that is degraded by any bodily motion such as cardiac motion, respiratory motion, motion produced by the musculoskeletal system, or gastrointestinal/genitourinary motion. Examples of these include external beam radiation therapy, placement of brachytherapy seeds, imaging test including but not limited to CT, MRI, fluoroscopy, ultrasound, and nuclear medicine, biopsies performed in any fashion, endoscopy, laparoscopic, thoracoscopic surgery, and open surgical procedures.

The environment surrounding a patient during a medical procedure presents unique challenges to any wireless localization system. For example, an operating room or doctor's office, includes various active sources of electromagnetic noise (e.g., overhead lighting, television, etc.) and reactive sources of electromagnetic noise that respond to a wireless exciter signal. In other words, other equipment can transmit noise that interferes with wireless localization systems. Examples include (a) active external noise caused by other electronic equipment that is broadcasting in the same frequency range being used by the wireless localization system; and (b) foreign RFID noise. Foreign RIFD noise is caused when the wireless localization system energizes RFID tags that are not intended or designed to be a part of the wireless localization system, triggering these foreign tags to respond with signals in the same frequency range being used.

Another challenge arises with changing environment materials. The environment may also include various magnetic, ferromagnetic, or metallic objects that may distort the magnetic fields generated and utilized by a wireless localization system. Eddy currents are generated within a conductor in response to an incident oscillating magnetic field, producing a field with opposite phase, which effectively produces a secondary signal source. The strength of the secondary source depends on the magnetic vector coupling between the primary emitter and the metal environment, which can be complicated and difficult to model. For example, operating rooms may include a bed upon which a patient is supported, and different beds will affect the magnetic fields differently. In another example, an operating room with a surgical robot may include various robotically controlled appendages or arms that may interfere or modify electromagnetic fields.

Improved systems and methods are needed for tissue and tool localization for medical procedures performed in a variety of environments.

### SUMMARY

The disclosure provides, in one aspect, a wireless localization system comprising a pad including an exciter coil and a sensor coil; and a tool including a wireless tag configured to generate a signal in response to a magnetic field generated by the exciter coil. The signal is detected by the sensor coil. The system further includes a processor configured to determine the location of the tool based on the signal detected by the sensor coil.

In some embodiments, the tool is one of a camera, an ultrasound probe, an electric impedance probe, a light probe, a microforce probe, an electrocautery tool, a needle, a swallowable capsule, a keypad, a stapler, a clamp, and a sponge.

In some embodiments, the wireless tag is a first wireless tag and the signal is a first signal, and wherein the system further includes a second wireless tag coupled to a tissue of a patient and configured to generate a second signal in response to the magnetic field generated by the exciter coil.

In some embodiments, the processor is configured to determine the location of the tool relative to the second wireless tag.

In some embodiments, the tissue that the second wireless tag is coupled to is one of a lung tissue, a bone tissue, a soft tissue, and an artery.

In some embodiments, the processor is further configured to determine the orientation of the tool.

The disclosure provides, in one aspect, a wireless localization system comprising a surgical robot assembly including a robotic arm, a camera, and a tool coupled to the robotic arm. The system further includes a pad including an exciter coil and a sensor coil. The system further includes a first wireless tag coupled to a portion of the surgical robot assembly. The first wireless tag configured to generate a first signal in response to a magnetic field generated by the exciter coil, and the first signal is detected by the sensor coil. The system further includes a second wireless tag coupled to a tissue of a patient, and the second wireless tag configured to generate a second signal in response to the magnetic field generated by the exciter coil. The second signal is detected by the sensor coil. The system further includes a processor configured to determine the location of the first wireless tag and the second wireless tag based on the first signal and the second signal detected by the sensor coil.

In some embodiments, the first wireless tag is coupled to the camera.

In some embodiments, the first wireless tag is coupled to the robotic arm.

In some embodiments, the sensor coil is a first sensor coil and the system further includes a second sensor coil coupled to the robotic arm.

In some embodiments, the system further includes a movable object including a third wireless tag, wherein the movable object is moved to various positions and detected by the camera to register the field of view of the camera.

In some embodiments, the movable object includes an outer shell, an inner sphere movable with respect to the outer shell. The third wireless tag is positioned within the inner sphere.

In some embodiments, the inner sphere includes a weighted portion to orient the sphere in a default orientation with respect to gravity.

In some embodiments, the surgical robot assembly includes a control console, and wherein the location of the first wireless tag and the location of the second wireless tag are displayed on the control console.

The disclosure provides, in one aspect, a pad comprising an exciter coil configured to generate a magnetic field, a sensor coil, an electrically conductive layer, and an electromagnetically permeable layer positioned between the exciter coil and the conductive layer.

In some embodiments, the electrically conductive layer is metallic and the electromagnetically permeable layer is ferrous.

In some embodiments, the electromagnetically permeable layer has a permeability within a range of 10 to 5000.

In some embodiments, the exciter coil is a first exciter coil and the pad further includes a second exciter coil, a third exciter coil, and a fourth exciter coil positioned circumferentially around a center.

In some embodiments, the magnetic field generated by the first exciter coil, the second exciter coil, the third exciter coil, and the fourth exciter coil comprises three orthogonal magnetic fields.

In some embodiments, the sensor coil is a first sensor coil, the pad further including a second sensor coil, a third sensor coil, and a fourth sensor coil.

In some embodiments, the first sensor coil, the second sensor coil, the third sensor coil, and the fourth sensor coil are positioned circumferentially around the first exciter coil.

In some embodiments, wherein the first sensor coil includes a first sensor axis and the third sensor coil includes a third sensor axis, and wherein the first sensor axis is parallel to the third sensor axis. The second sensor coil includes a second sensor axis and the fourth sensor coil includes a fourth sensor axis, and wherein the second sensor axis is parallel to the fourth sensor axis.

In some embodiments, the first sensor axis is perpendicular to the second sensor axis.

In some embodiments, the first exciter coil includes an exciter coil axis perpendicular to the first sensor axis and the second sensor axis

In some embodiments, the sensor coil detects a wireless signal in response to the magnetic field generated by the exciter coil, and wherein the pad is positioned between a patient and a bed supporting the patient.

The disclosure provides, in one aspect, a wireless tag comprising an outer housing including an anchor, wherein the anchor is configured to be secured within a tissue of a patient.

In some embodiments, the anchor is self-deploying.

In some embodiments, the anchor is a spiral.

In some embodiments, the anchor is a stent.

In some embodiments, the anchor extends radially outward from a longitudinal axis of the outer housing.

Other aspects of the disclosure will become apparent by consideration of the detailed description and accompanying drawings.

### DEFINITIONS

As used herein, the terms “processor” and “central processing unit” or “CPU” are used interchangeably and refer to a device that is able to read a program from a computer memory (e.g., ROM or other computer memory) and perform a set of steps according to the program. As used herein, the term “processor” (e.g., a microprocessor, a microcontroller, a processing unit, or other suitable programmable device) can include, among other things, a control unit, an arithmetic logic unit (“ALC”), and a plurality of registers, and can be implemented using a known computer architecture (e.g., a modified Harvard architecture, a von Neumann architecture, etc.). In some embodiments the processor is a microprocessor that can be configured to communicate in a stand-alone and/or a distributed environment, and can be configured to communicate via wired or wireless communications with other processors, where such one or more processor can be configured to operate on one or more processor-controlled devices that can be similar or different devices.

As used herein, the term “memory” is any memory storage and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processor can be connected to the memory and execute software instructions that are capable of being stored in a RAM of the memory (e.g., during execution), a ROM of the memory (e.g., on a generally permanent bases), or another non- transitory computer readable medium such as another memory or a disc. In some embodiments, the memory includes one or more processor-readable and accessible memory elements and/or components that can be internal to the processor-controlled device, external to the processor- controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the processor can be configured to retrieve from the memory and execute, among other things, instructions related to the processes and methods described herein.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks, whether local or distant (e.g., cloud-based).

“About” and “approximately” are used to provide flexibility to a numerical range endpoint by providing that a given value may be “slightly above” or “slightly below” the endpoint without affecting the desired result.

The term “coupled,” as used herein, is defined as “connected,” although not necessarily directly, and not necessarily mechanically. The term coupled is to be understood to mean physically, magnetically, chemically, fluidly, electrically, or otherwise coupled, connected or linked and does not exclude the presence of intermediate elements between the coupled elements absent specific contrary language.

As used herein, the term “in electronic communication” refers to electrical devices (e.g., computers, processors, etc.) that are configured to communicate with one another through direct or indirect signaling. Likewise, a computer configured to transmit (e.g., through cables, wires, infrared signals, telephone lines, airwaves, etc.) information to another computer or device, is in electronic communication with the other computer or device.

As used herein, the term “transmitting” refers to the movement of information (e.g., data) from one location to another (e.g., from one device to another) using any suitable means.

As used herein, the term “network” generally refers to any suitable electronic network including, but not limited to, a wide area network (“WAN”) (e.g., a TCP/IP based network), a local area network (“LAN”), a neighborhood area network (“NAN”), a home area network (“HAN”), or personal area network (“PAN”) employing any of a variety of communications protocols, such as Wi-Fi, Bluetooth, ZigBee, etc. In some embodiments, the network is a cellular network, such as, for example, a Global System for Mobile Communications (“GSM”) network, a General Packet Radio Service (“GPRS”) network, an Evolution-Data Optimized (“EV-DO”) network, an Enhanced Data Rates for GSM Evolution (“EDGE”) network, a 3GSM network, a 4GSM network, a 5G New Radio, a Digital Enhanced Cordless Telecommunications (“DECT”) network, a digital AMPS (“IS-136/TDMA”) network, or an Integrated Digital Enhanced Network (“iDEN”) network, etc.

As used herein, the term “subject” or “patient” refers to any animal (e.g., a mammal), including, but not limited to, humans, non-human primates, companion animals, livestock, equines, rodents, and the like, which is to be the recipient of a particular treatment. Typically, the terms “subject” and “patient” are used interchangeably herein in reference to a human subject.

As used herein, the term “subject/patient suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass) but for whom the stage of cancer is not known. The term further includes people who once had cancer (e.g., an individual in remission).

As used herein, the term “biopsy tissue” refers to a sample of tissue (e.g., breast tissue) that is removed from a subject for the purpose of determining if the sample contains cancerous tissue. In some embodiments, biopsy tissue is obtained because a subject is suspected of having cancer. The biopsy tissue is then examined (e.g., by microscopy; by molecular testing) for the presence or absence of cancer.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include tissue, blood products, such as plasma, serum and the like. Such examples are not however to be construed as limiting the sample types applicable to the present invention.

As used herein, the term “tag,” “marker tag,” “wireless tag,” or “SmartClip®” refers to the small implantable marker that, when excited by an exciter's time varying magnetic field, will emit a “homing beacon” spectrum of frequency(ies) received by the “sensor coil(s)” or “witness coil(s)” and used to determine its location. It may be programmed to produce a unique spectrum, thus permitting multiple tags to be implanted and located simultaneously.

### BRIEF DESCRIPTION OF THE DRAWINGS

FIG. 1 is a top view schematic of a wireless localization system include a wirelessly tracked tool in an operating room for a medical procedure.

FIG. 2 is a schematic view of a needle including a wireless tag and a patch with a wireless tag coupled to a patient skin.

FIG. 3A is a schematic view of a keypad and stylus with wireless tags.

FIG. 3B is an illustration of a localized probe and localized needle with visual markings.

FIG. 4 is a top view schematic of a wireless localization system including a wirelessly tracked surgical robotic system in an operating room for a medical procedure.

FIG. 5 is a side view schematic of a robotic arm with wireless tags and a sensor coil.

FIG. 6A is a schematic view of a movable object for registering a camera view.

FIG. 6B is a method for registering a camera to a wireless localization system.

FIG. 7 is a side view of a wireless localization tag including a plurality of tines.

FIG. 8 is a perspective view of a wireless tag including a plurality of self-deploying tines.

FIG. 9 is a perspective view of a wireless tag including an anchor.

FIG. 10 is a perspective view of a wireless tag including an anchor.

FIG. 11 is a schematic of a pad including four exciter coils generating three orthogonal magnetic fields.

FIG. 12 is a perspective view of a cross-section of a pad including an electromagnetically permeable layer and an electrically conductive layer.

FIG. 13 is a top view of a pad including four exciter coils and twelve sensor coils.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In the foregoing description of preferred embodiments, specific terminology has been resorted to for the sake of clarity. However, the invention is not intended to be limited to the specific terms so selected, and it is to be understood that each specific term includes all technical equivalents which operate in a similar manner to accomplish a similar technical purpose. Terms such as “top” and “bottom”, “front” and “rear”, “inner” and “outer”, “above”, “below”, “upper”, “lower”, “vertical”, “horizontal”, “upright” and the like are used as words of convenience to provide reference points.

Provided herein are systems, devices, assemblies, and methods for integrating a remotely located tag into medical procedures. While the specification focuses on medical uses in human tissues, it should be understood that the systems and methods find broader use, including non-human uses (e.g., use with non-human animals such as livestock companion animals, wild animals, or any veterinary settings). For example, the systems may be used in environmental settings, agricultural settings, industrial settings, or the like.

### A. Wireless Tool Tracking Applications

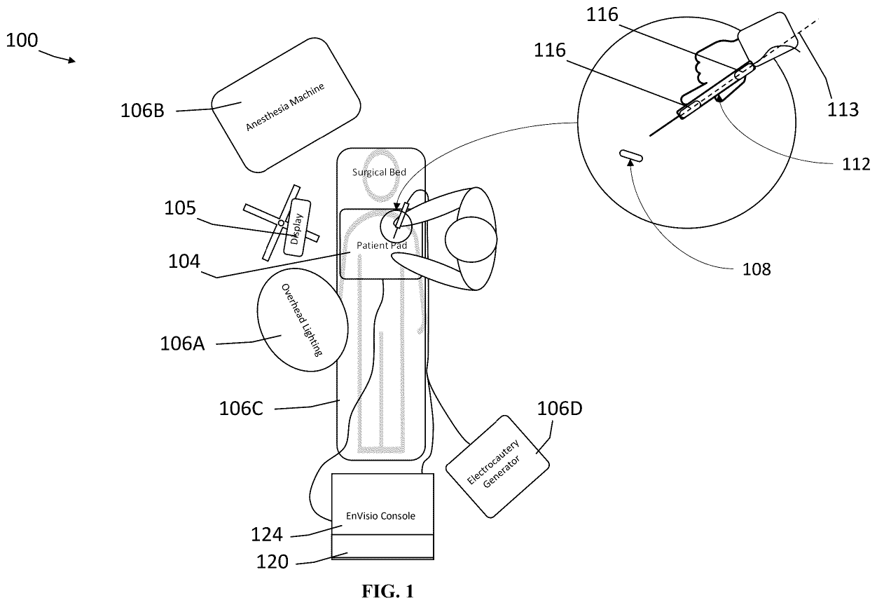

In addition to being located within human tissue, the tags can be integrated into tools to wireless track the location and orientation of tools utilized in various medical procedures. With reference to FIG. 1, a wireless localization system 100 is schematically shown in an operating room (i.e., a doctor's office, a surgical suite, etc.) for a general medical procedure. The wireless localization system 100 includes a pad 104 positioned underneath a patient. In some embodiments, the pad 104 includes at least one exciter coil (e.g., exciter) and at least one sensor coil (e.g., witness station). A wireless tag 108 is coupled to a tissue of a patient and is configured to generate a wireless signal in response to a magnetic field generated by the exciter coil. In some embodiments, the wireless tag 108 is configured to generate a plurality of wireless signals (e.g., a first wireless signal and a second wireless signal) in response to a magnetic field generated by the exciter coil. As discussed further herein, the wireless tag 108 is coupled to tissue (e.g., lung tissue, bone tissue, soft tissue, an artery, etc.) depending on the medical procedure. Examples of such a wireless tag 108 are described in U.S. patent application Ser. No. 15/113,703 and is incorporated herein by reference in its entirety.

With continued reference to FIG. 1, a tool 112 includes at least one wireless tag 116 that is configured to generate a wireless signal in response to a magnetic field generated by the exciter coil of the pad 104. The wireless signal from the wireless tags 116 in the tool 112 and the wireless tag 108 in/on patient are detected by the sensor coil, for example, in the pad 104. As detailed herein, the tool 112, in some embodiments, is a surgical tool with wireless tags 116 embedded therein.

A processor 120 (shown in FIG. 1 as part of a wireless localization console 124) is configured to determine the location of the tool 112 and the location of the targeted area in the patient based on the signals from the wireless tags 108, 116 and measured by the sensor coil. As discussed further herein, the relative position of the tag 108 in the patient tissue and the tool 112 is utilized to improve the outcome of medical procedure. For example, the relative distance between the tag 108 and the tool 112 may be displayed visually (e.g., on a display 105) or by providing audio or haptic feedback to a user. In some embodiments, the system 100 is tracking the location of a plurality of wireless tags.

In the illustrated embodiment, the tool 112 includes two wireless tags 116 positioned along a longitudinal axis 113 of the tool 112 to determine the orientation of the tool 112 in addition to the position of the tool 112. In some embodiments, the tags 116 are positioned within an outer housing of the tool 112. In other embodiments, the tags 116 are positioned on an outer surface of the tool 112.

With continued reference to FIG. 1, the environment in which the wireless localization system 100 is positioned includes various active and reactive sources of electromagnetic noise. For example, the environment in FIG. 1 includes overhead lighting 106A, an anesthesia machine 106B, a display 105, a surgical bed 106C, and an electrocautery generator 106D. Each of which may affect the electromagnetic field present around the wireless localization system 100. As described herein, the wireless localization system 100 effectively tracks (e.g., localizes) a plurality of wireless tags in proximity to various active and reactive sources of electromagnetic noise

In some embodiments, the tool 112 is a camera. For example, some medical procedures utilize a camera to visualize the treatment area, but wireless tags implanted in a patient are not typically visible in the camera view. A solution presented herein is a camera with an integrated wireless tag to track the location of the camera and a processor to determine a coordinate transformation from the implant tracker coordinates to the camera field of view coordinates. As such, the location of the implanted tag (e.g., tag 108) can be superimposed on the camera view that is presented to the user (e.g., the display 105)—thereby improving a user's identification of the tag from the camera's field of view.

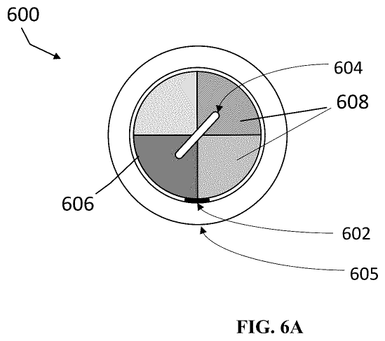

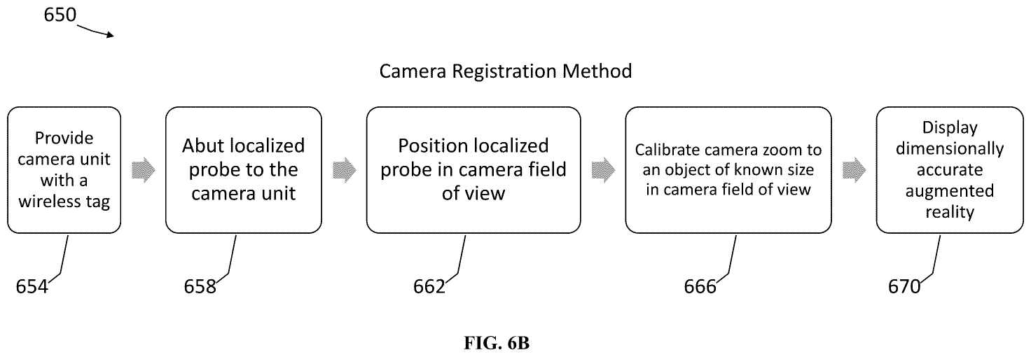

To determine the coordinate transformation, an object of known geometry is simultaneously imaged with the camera and localized with the wireless localization system. To track the camera, a wireless tag (e.g., tag 116) is fixed to the camera and also tracked with the wireless localization system. To track roll of the camera, or if the wireless tag cannot be co-axial with the camera, two wireless tags are utilized. Registration of the camera can occur in two ways: either the camera remains stationary and a moving object is tracked (see FIG. 6A), or a stationary object is registered to a moving camera. In some embodiments the camera is manually moved by a user. In other embodiments, the camera is moved by an actuator.

In some embodiments, the tool 112 is an ultrasound probe. Although ultrasound can be used to interrogate tissue and determine the position of target tissue, the imaging is two-dimensional and a separate probe must be used. A solution presented herein is to track the position and orientation of the ultrasound probe, simultaneously with the position of an implanted tag (e.g., tag 108). Ultrasound images at many positions and orientations may be acquired. Then, swap the ultrasound probe with a tool used to perform surgical excision, which is also tracked. Next, the ultrasound images are replayed according to the position of the electrosurgical tool, corrected by any relative shifts in implant position. Furthermore, ultrasound images can be segmented by tissue type, and a 3D model built up, registered to the implant position. After enough images are built up, the 3D model can be maintained relative to the implant position and used in live navigation.

In some embodiments, the tool 112 is an electrical impedance probe. It may be helpful to measure the 3D contours of the patient body including any external surfaces and internal organs (i.e., measure the geometry of excised tissue). A solution presented herein utilizes an electrical impedance probe to detect when the electrical impedance probe is in contact with the patient tissues. If the location of the probe tip is tracked the geometry of any part of the patient can be acquired by moving the probe tip around the skin.

In some embodiments, a tracked probe provides an ultrasound of patient tissue that is used to determine the location of a target with respect to tags positioned on the skin surface (e.g., embedded within an adhesive material adhered to a patient's skin). Once the ultrasound is segmented, direct feedback to the user can be provided on the needle placement, insertion orientation, and depth of insertion. In some embodiments, a motorized needle guide is tracked and adjusted to ensure proper needle insertion.

In some embodiments, the tool 112 is a light probe. Spectroscopy can be performed, for example, by illuminating tissue with broadband light and collecting reflected light but keeping track of the position of data collection must be performed manually. A solution presented herein collects the position and spectra data simultaneously, such that the location of anomalies or features of interest can be automatically collected and presented to the user.

Lung surgeons need the location of the tumor to be clearly visible during video-assisted thoracis surgery (VATS) and robot assisted thoracis surgery (RATS) procedures. Surgeon attempt position 2D or 3D cameras to define a point of view, and conventional video processing can overlay graphics and text on the video feed provided by the user. But video processing requires interconnections between systems, typically by cables, software, and site-specific configurations. A solution presented herein is projected light that overlays graphics and text on the tissue within the camera field of view. Alternatively, a tracked probe in the camera view can be equipped with one or more controlled light source(s).

A projected light implementation compatible with VATS and RATS procedures, in some embodiments, includes a robotically controlled laser pointer mounted near the camera, where two motor controlled axial degrees of freedom allow the system to always point the laser at the wireless tag, thereby illuminating the location on the tissue surface directly between the camera and the wireless tag. In some embodiments, the distance from the tissue surface to the wireless tag is determined by touching a wireless tracked probe to the tissue at the point of illumination, or by using the camera video feed to determine the location of the point of illumination. In such an embodiment, the camera is calibrated and tracked within the same reference frame as the wireless tag and light projector. In some embodiments, the projected light and machine vision processing is configured for structured light projection to determine tissue geometry more accurately. In some embodiments, rasterization is used to project other graphics and text. In some embodiments, the light projection technique has no moving parts. In some embodiments, a high-powered light is utilized to burn the tissue to create a durable mark on the tissue.

A tracked probe with a controlled light source compatible with VATS and RATS procedures, in some embodiments, includes a single light emitting diode (LED) attached to the tracked probe. In some embodiments, the LED brightness is controlled by the system based on the relative position and/or alignment of the probe to the wireless tag. For example, the user could manipulate the tracked probe until the user sees the illumination in the camera view, and when the brightness is maximized, the tracked probe is pointed directly at the wireless tag. In other embodiments, a tracked probe with a controlled light source compatible with VATS and RATS procedures includes multiple LEDs arranged in a pattern. In some embodiments, each LED brightness is controlled by the system based on the relative position and/or alignment of the probe to the wireless tag. For example, the user could observe the pattern that is configured to guide them to the wireless tag. In some embodiments, a set of LEDs arranged in a plane is lighted so that the location of the highest intensity corresponds to the position of the wireless tag.

In some embodiments, the tool 112 is a microforce probe. The clamping force of a robot may be easy to control, but there are advantages to also wirelessly monitoring the clamping force. In some instances, a robotic jaw is configured to grab an object, but there is no feedback on how much resistance the robotic jaws are experiencing. The solution presented herein is a wireless tag embedded into a small module with a wireless communications module and one or more sensors and mechanical interfaces to measure and transmit real time data such as force, temperature, pressure, etc. In some embodiments, the tool 112 is a specialized tracked tool that deflects in a way that is measurable via the angle between two wireless tags, and the deflection is related to the amount of force applied to what is captured within the jaws. Advantageously, no battery is required because the communications can be powered by the exciter via induction. In other words, the same exciter used for localization can also provide power to the microsensor. As such, size can be minimized. For example, communications chips can be as small as approximately 2.5 mm×approximately 2.5 mm without an antenna. The probe data can be utilized, for example, to generate haptic feedback to the user.

In some embodiments, the tool 112 is an electrocautery multi-tool. Conventional electrocautery tools include various cords or tubes that must be assembled by the user. A solution presented herein is a single device with smoke capture, illumination, electrocautery, and tip localization. The solution consolidates multiple cords into one and eliminates user assembly steps. It is beneficial for the localization and electrocautery system to be coordinated so that interference is mitigated. Advantageously, better cutting energy can be delivered by controlling the waveforms more precisely, and by using low energy to detect when the tip is in contact with tissue. In addition, low energy detection of the tip in contact with tissue can be used to build a 3D model of the patient.

In some embodiments, the tool 112 is an electrocautery tool accessory (e.g., a collar, a tip, etc.). Conventional electrocautery tools include a tip removable from the pen. A solution presented herein is an adaptor for an electrocautery tool (e.g., a bovie pen). In some embodiments, the adaptor is a small wireless device that attaches to the electrocautery tool like a collar. In other embodiments, a locatable tip (i.e., a tip with wireless tag embedded in the shaft) is provided such that the tip can be tracked wirelessly by the wireless localization system.

In some embodiments, the tool 112 is a stapler. In lung applications, a cut is optimally performed perpendicular to the lung plane. A solution presented herein is a stapler including at least one wireless tag configured to define the plane where the lung will be cut and the end of the stapler. In some embodiments, the wireless tag is oriented perpendicular to the plane of interest, which aligns an unknown roll coordinate of the wireless tag to be anywhere on the plane.

In some embodiments, the tool 112 is a tracked clamp configured to span the entire cut across a lung.

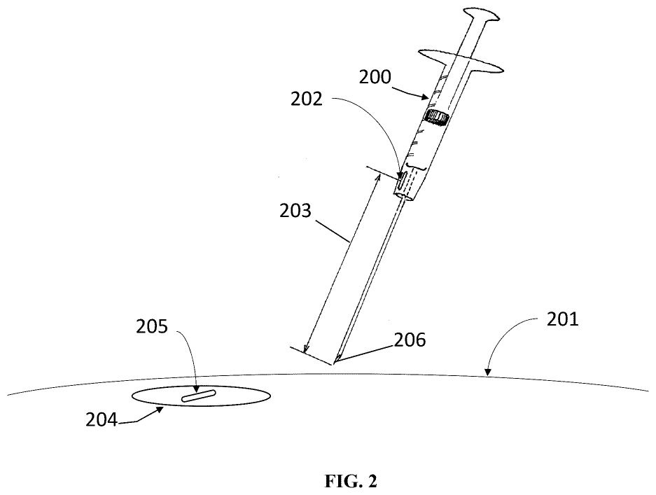

In some embodiments, the tool 112 is a needle that is utilized in a medical procedure. A solution presented herein includes a needle 200 with a wireless tag 202 (e.g., a SmartClip®) in the upper shaft that can be tracked. With reference to FIG. 2, the needle 200 includes the wireless tag 202 coupled to the needle 200 for localization of the needle 200. In some embodiments, a patch 204 including a wireless tag 205 is positioned on the skin of a patient 201 to localize the skin surface (e.g., to track location of skin surface). A known distance 203 between the wireless tag 202 and a tip 206 of the needle 200 is utilized to provide advantageous information. As such, the needle depth of insertion is tracked by the patch 204 that is coupled to the patient skin near an intended punction site. Also, the position and orientation of the patch 204 would allow the angle of the needle 200 with respect to the patient 201 to be calculated. Alternatively, the patch 204 is eliminated and the depth of insertion is tracked by having the user indicate when the tip 206 of the needle 200 is at the skin surface 201.

In some embodiments, the tool 112 is a capsule or pill that is swallowed by a patient. For example, convention capsules are swallowed by a patient to diagnose GI issues. A solution presented herein is a capsule with integrated wireless tags to provide wireless tracking of the capsule to increase the utility of the devices. The capsule with an integrated wireless tag can be enabled to deliver medication based on a command given over wireless communication. Medication delivery could be protected by requiring a large pulse of energy from an exciter. The large pulse can physically change the capsule between a medication deliver prohibited configuration to a medication delivery allowed configuration. In addition, a capsule with an integrated tag could implant a wireless tag on demand to mark tissue of interest.

In some embodiments, the tool 112 is a keypad and stylus (i.e., a sterile surgical interface). In surgery, sterile users cannot touch non-sterile user input devices. Bringing additional wired devices to serve as input devices requires additional sterile field management. Conventional wireless input devices are typically expensive and require batteries. The solution presented herein provides a keypad without electronics or digital communications with an embedded wireless tag that can be localized (i.e., a low-cost keypad). A localized stylus or electrocautery tool can touch the localized keypad.

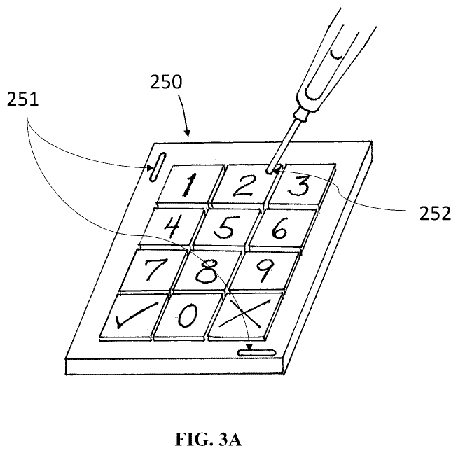

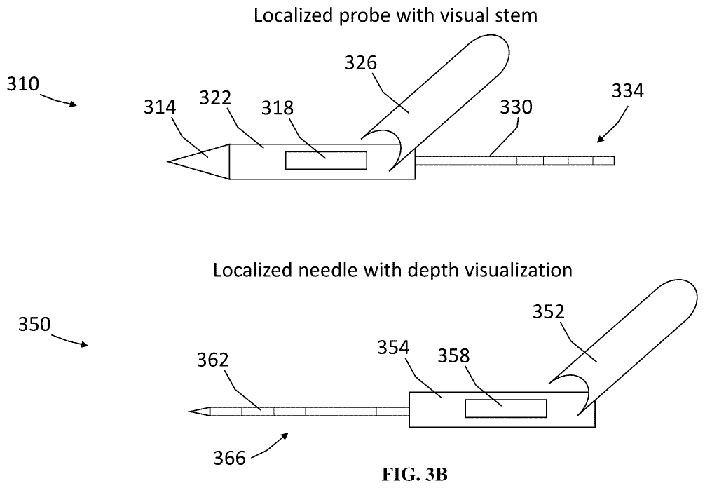

With reference to FIG. 3A, a keypad 250 includes two wireless tags 251 and is localized by a wireless localization system (e.g., system 100). The system then determines which key on the keypad is touched by tracking the location of a tracked tool tip or stylus 252 with respect to the keypad 250. In other words, the tracked tool tip 252 location is known and the location of the buttons are known with respect to the wireless tags 251 in the keypad 250. As such, the localization system can detect the location of the tracked tool tip 252 and will register a button press by the tool tip 252 relative location to the keypad 250. In some embodiments, the keypad provides a mechanical response on each button which can be recognized by the localization system as a “click” based on the characteristic movement of the tracked tool (e.g., tracked stylus, tracked electrocautery tool, etc.). In some embodiments, the keypad is located by positioning a stylus with a single wireless localization tag at or on the keypad.

With reference to FIG. 3B, in some embodiments, the tool 112 is a localized probe 310 that is graspable by a robotic arm or tool 326. The localized probe 310 includes a conical tip 314 and a wireless tag 318 positioned within a housing 322. In the illustrated embodiment, the housing 322 is graspable by the robotic arm or tool 326. The probe 310 further includes a stem 330 with indicia 334 (e.g., visual markings) that can be detected and tracked by a camera. In other words, the localized probe 310 is a small wirelessly tracked probe that can be grasped by a robot or other instrument within the patient body, where that probe has a stem that acts as a visual reference. In some embodiments, the orientation of the probe 310 is constrained to define the orientation of the probe. In other words, the robotic arm 326 can be constrained to always keep the probe 310 oriented vertically, with the stem 330 pointing upwards.

With continued reference to FIG. 3B, in some embodiments, the tool 112 is a localized needle 350 that is graspable by a robotic arm or tool 352. The localized needle 350 includes a housing 354 with a wireless tag 358 and a needle portion 362 with indicia 366 (e.g., visual markings) that can be detected and tracked by a camera. In other words, the localized needle 350 includes an insertion depth dimensions readable by a camera. As such, in some embodiments, the localized probe is inserted into the patient organ until its tip is co-located with an implanted wireless tag so that it functions as a physical landmark visualized by a camera. In some embodiments, it could be tactilely sensed.

### B. Robotic Integration Applications

As disclosed herein, wireless tags are integrated into robots and robotic apparatuses utilized in various medical procedures. An example of such a robotic system is the Intuitive da Vinci System. An example of a computer-assisted tele-operated surgery system and methods disclosed in U.S. Pat. No. 11,207,143, the entire contents of which are incorporated herein by reference. Unique challenges arise when attempting to integrate a wireless located tag into a robotic environment.

For example, patient anatomy and pre-operating imaging is insufficient for many surgical interventions. Wired-only navigation systems depend on pre-operative imaging and other methods to register detected locations to patient anatomy. As mentioned, some medical procedures utilize a camera, but wireless tags implanted in a patient are not typically visible in the camera view. A solution presented herein provides wireless tag localization in a robotic application. Precise localization of an implanted tag, co-registered to the surgeon's visual frame of reference, along with localization of tools, can provide intuitive dimensional feedback essential for successful interventions like excision, ablation, drug delivery, etc.

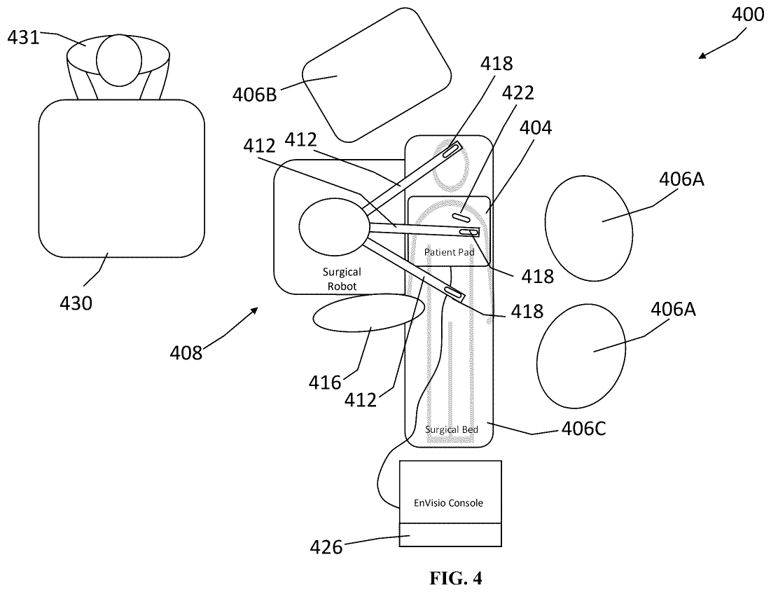

With reference to FIG. 4, a wireless localization system 400 is schematically shown in an operating room (i.e., a doctor's office, a surgical suite, etc.) for a general medical procedure. The environment in which the wireless localization system 400 is positioned includes various active and reactive sources of electromagnetic noise. For example, the environment in FIG. 4 includes overhead lighting 406A, an anesthesia machine 406B, and a surgical bed 406C. Each of which may affect the electromagnetic field present around the wireless localization system 400. The wireless localization system 400 includes a pad 404 positioned underneath a patient. In some embodiments, the pad 404 includes at least one exciter coil and at least one sensor coil (e.g., witness station). The wireless localization system 400 includes a surgical robot assembly 408 (e.g., a da Vinci System developed by Intuitive). In the illustrated embodiment, the surgical robot assembly 408 includes a robotic arm 412, a camera 416, and a tool coupled to the distal end of the robotic arm 412. In some embodiments, the surgical robot assembly 408 includes a single robotic arm. In other embodiments, the surgical robot assembly 408 includes at least one robotic arm. In the illustrated embodiment, the surgical robot assembly 408 includes three robotic arms 412. In some embodiments, the surgical robot assembly 408 includes a plurality of cameras. As explained in greater detail herein, the wireless localization system 400 integrates the surgical robot assembly 408 to provide improved localization of portions of the surgical robot assembly 408 relative to a target area in the patient. For example, visualization of a wireless tag 422 embedded within a patient can be visualized and overlayed on the robotic vision from the camera 416. In some embodiments, the surgical robot assembly includes a control console 430 where a surgeon 431 controls the robot and the location(s) of the wireless tags are displayed on the control console. In some embodiments, the control console 430 is in the same location (e.g., room) as the surgical robot and in other embodiments, the control console 420 is in a different location (e.g., a remote location, offsite location).

With continued reference to FIG. 4, a first wireless tag 418 is coupled to a portion (e.g., a robotic arm) of the surgical robot assembly 408, and a second wireless tag 422 is coupled to a target tissue of a patient. The first wireless tag 418 and the second wireless tag 422 are configured to generate wireless signals in response to a magnetic field generated by the at least one exciter coil in the pad 404. The wireless signals from the tags 418 and 422 are detected by the at least one sensor coil in the pad 404. A processor 426 then determines the location of the first wireless tag 418 and the second wireless tag 422 based on the signals measured by the at least one sensor coil.

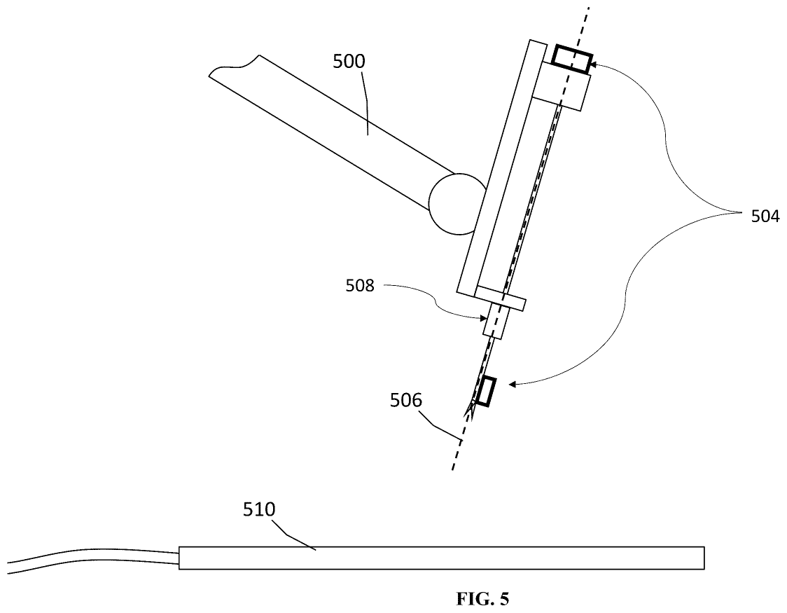

With reference to FIG. 5, in some embodiments the each of the robotic arms 500 include two wireless tags 504 mounted along a longitudinal axis 506 aligned with the surgical tool mounted on a distal end of the arm. In some embodiments, a sensor coil 508 (similar to and/or in addition to the sensor coils in the patient pad 510) is coupled to the robot arm 500 in order to improve the sensing of wireless tag signals that may be poisoned far away from the patient pad 510. In the illustrated embodiment, the sensor coil 508 is positioned axially between the two wireless tags 504, along the longitudinal axis 506. In some embodiments, the sensor coil 508 is oriented orthogonal to the coils in the wireless tags 504.

One challenge to integrating wireless tags into robotic applications is effectively using cameras to measure anatomical landmarks. Use of camera to measure landmarks has drawbacks such as, challenges with lighting, lack of clear reference marks, tissue changes compared to as-imaged, presence of fluids, etc. A solution presented herein allows for co-registration to be achieved by registering the camera location with respect to wireless tags mounted on the camera and by tracking an object that goes to specific points in the camera's field of view. A wireless tag mounted to the camera is not mounted directly on the lens, so the relative position of the wireless tag and the lens of the camera is registered to improve localization. In some embodiments, field of view registration is accomplished by moving a tracked object to the upper-right, lower-right, upper-left and lower-left corners of the field of view when the position of the camera is known. In some embodiments, the camera is calibrated by placing a first wireless tag at a known close distance from the camera (e.g., within approximately 50 mm) to get the camera position, and then the orientation of the camera is determined by imaging a second wireless tag with the camera while the second wireless tag is far away from the camera. The wireless localization system can locate the magnetic center and the direction of the longitudinal axis of the wireless tag, but not necessarily which end of the tag is which. In order to consistently co-register the camera view to the system coordinate system, an additional degree of freedom is required to be constrained.

With reference to FIG. 6A, a tracked object 600 for camera registration is illustrated. The tracked object 600 includes a clear outer shell 605 and an inner sphere 606 that is freely movable with respect to the outer shell 605. In some embodiments the outer shell 605 is configured to be gripped or grabbed by a robotic tool. A wireless tag 604 is positioned within the inner sphere 606. In the illustrated embodiment, a weighted portion 602 (e.g., weighted bottom) uses gravity to ensure a wireless tag 604 in an inner sphere 606 always “points” a given direction (i.e., upwards and away from the patient pad) and thereby provides enough information to constrain the last degree of freedom. In other words, the inner sphere 606 includes a weighted portion 602 to orient the inner sphere 606 and the tag 604 in a default orientation with respect to gravity. The inner sphere 606 further includes a plurality of sections 608 of various colors or shades of black and white that are detectable by the camera. In the illustrated embodiment, the plurality of sections 608 includes four different color sections. In some embodiments the plurality of sections 608 includes eight different color sections. As such, the movable tracked object 600 includes the wireless tag 604 and is moved to various positions and detected by the camera to register the field of view of the camera

In some embodiments, the wireless tags are cylindrical and the roll about the longitudinal axis and the direction the tag is pointing are challenging to determine. A solution presented herein is to place a wireless tag within an asymmetrically weighted mount. The wireless tag in the mount creates an angle of approximately 20-70 degrees from a line extending between the mount's center of mass and the mount's axis of rotation. The asymmetric weight provides a constraint on the tag orientation sufficient to allow the determination of both the roll orientation and the pointing direction. In some embodiments, the mount is encased in an object having a known pattern (e.g., the plurality of sections 608). The known pattern is imaged with a camera, allowing the object to be simultaneously localized with a camera and the wireless localization system. Once simultaneously localized, the general transformation between the wireless localization system and the camera is determined.

If the position of the camera is not known, the four corners of the field of view can be measured at two different distances from the camera to determine the camera location. In some embodiments, the camera performs a set of pre-determined motions (e.g., spin, move forward, etc.). In other embodiments, the camera looks at the same point from different points of view. In some embodiments, configurations of the camera registration can be saved and loaded as a pre-set configuration.

With reference to FIG. 6B, a method 650 is disclosed to register a camera to a wireless localization system. The method 650 includes a STEP 654 of providing a camera unit with a wireless tag mounted to the camera unit. The method 650 also includes a STEP 658 of moving a localized probe to contact the camera unit. In other words, at the STEP 658 includes abutting a localized probe to the camera unit. The method 650 also includes a STEP 662 to position the localized probe in the camera field of view. In some embodiment, the localized probed position in the field of view is the same probe from STEP 658. In other embodiments, the localized probe positioned in the view of view is different from the localized probe used in STEP 658. The method 650 further includes a STEP 666 to calibrate the camera zoom utilizing an object of a known size that is positioned within the camera field of view. In some embodiments, a calibration outline is displayed to a user and the camera zoom is adjusted until the object of known size is contained within the calibration outline. In one embodiment, a known object with a 10 cm diameter is positioned within the camera field of view and the zoom on the camera is adjusted until the object fits within a calibration outline on the display. The method 650 further includes a STEP 670 of displaying a dimensionally accurate augmented reality. In other words, at the STEP 670, the camera field of view can be overlayed with a 3D interface. In some embodiments, two 3D interfaced are provided on separate video feeds with one to each eye so that the localization interface is overlayed on top of a 3D camera interface. As such, the method 650 provides a calibration process to register a camera view to a wireless localization system that visualizes the location of various wireless tags. In some embodiments, additional corrections are applied to account for various camera lens types (e.g., wide aperture lens adjustments).

Another challenge to integrating wireless tags into a surgical robot application is that metal object should be kept away from detectors or sensor coils because they create distortions in the electromagnetic field. In other words, surgical robots often include metal components (e.g., a metallic arm) that can distort the electromagnetic field relied upon by the wireless localization system. A solution presented herein is to utilize various noise reduction and signal processing techniques. In some embodiment, the system utilizes a phase sensitive signal processing.

Another challenge to integrating wireless tags into a surgical robot application is attaching wireless tags directly to metal components (e.g., a robotic arm) can cause the signal transmitted by the wireless tag to degrade by induced eddy currents within the metal. A solution presented herein is to include a thin layer of high permeability material (e.g., iron, manganese, zinc, silicon, aluminum, nickel, electrical steel, cobalt-iron, etc.) positioned between the antenna winding of the wireless tag and the metal. In some embodiments, the layer of high permeability material is selected based on the frequency range of the signal transmitted by the wireless tag.

Another challenge to integrating wireless tags into a surgical robot application is that range is limited by the ability to measure and a feature of interest on a patient can often be positioned far away from the pad. The solution presented herein is to embed a high gain detector (e.g., 508 of FIG. 5) orthogonal to emitters to sense the wireless tag signals. The high gain detector can be mounted on a robotic component (e.g., a robot arm) and the sensor moved close to the wireless tag.

Another challenge to integrating tags into a surgical robot application is that small wireless tags respond to only one direction of the excited field, and in some embodiment the robotic applications would have a plurality of wireless localization tags (i.e., clips, SmartClips®) being tracked. A solution presented herein utilizes a pad that creates three orthogonal directions of an exciter field. With reference to FIG. 11, an exciter pad 1102 is illustrated generating three electromagnetic fields just above the pad 1102 that are orthogonal to each other (e.g., X, Y, and Z direction electromagnetic fields). With three orthogonal directions of the exciter field, there is enough power transfer for wireless tags regardless of the wireless tag orientation. The preferred field direction for any wireless tag can be determined by measuring the total power received by the sensing system and choosing the field direction that has the largest total power. If multiple tags need to be localized, only those excited by the same field direction can be localized at the same time. By sequentially stepping through the minimum number of field directions which cause all wireless tags to be localizable, all tags are localized. The amount of time it takes to change field direction is important. To minimize the time, solid-state transistors are used to change the field polarity (in place of, or in addition to, electromechanical relays). In some embodiments, the electromechanical relays may take longer to switch than the solid-state transistors and electromechanical relays do create noise at the time of switching that must be allowed to decay out of the narrow bandwidth signal conditioning. An example pad with an exciter coil and a sensor coil is described in U.S. Pat. No. 10,278,779, the entire contents of which are incorporated herein by reference.

In some embodiments, surgical robots perform an automated intervention (e.g., a set of steps based on a detailed surgical plan for robotic movement). Another challenge to integrating tags into robotic applications is that automated interventions could be based on a detailed surgical plan for robotic movements. A solution presented herein is automation safety that is enhanced by monitoring the robot movements by an independent processing system that compares the actual movements to the planned movements, and generates a signal when the plan diverges beyond allowed tolerances. A secondary signal is generated when the robot movements are not being tracked. In other words, in some embodiments, the wireless tags are utilized to track and double check the robotic components are moving according to the plan. As such, the wireless tags can wirelessly track the movement of robotic components and may be utilized to provide additional safety or a safety lockout.

### C. General Surgical Applications

The solutions provided herein also have general surgical applications including, but not limited to: preventing items being left within patients, bone modeling, and soft tissue modeling.

A general problem with surgery arises when items (e.g., surgical instruments, sponges, etc.) are left within a patient after surgery is completed. The solution presented herein embeds wireless tags into such items so that they can be tracked wirelessly. Conventional fielded wireless object tracking system are incompatible with the wireless tag localization system. As described herein, fielded wireless object tracking is integrated into the tag localization system to reduce the amount of equipment required and/or reduce the amount of wireless interference. Items tagged with a tag are detectable with high assurance with the En Visio® system. A large number of tags can be supported if localization requirements are relaxed. In some embodiments, the system keeps an ongoing count and tracks the number of items introduced to the general fielded area and decrease the number when tagged items leave the general fielded area (i.e., keeps a running total of items in the environment).

Another challenge is conventional robot-guided orthopedic systems rely on optical tracking of bones, which requires optical trackers to be surgically implanted into bone and that the system maintains direct line of sight of the optical trackers. The solution presented herein embeds two or more wireless tags (i.e., SmartClips®) into a bone, use pre-operative imaging and image segmentation to establish the bone geometry within the coordinate system defined by two or more wireless tags. In some embodiments, at time of surgery the bone is mounted to motor driven actuators controlled by software (e.g., a robotic arm), and the bone interface includes the wireless tag excitation and sensing sub-systems.

Another challenge is that pre-operative imaging is collected with soft tissue in different positions, orientations, and shape than it is presented in the operating room for surgery. The solution presented herein is to implant two or more wireless tags within soft tissue and/or on the skin of the patient. Pre-operating imaging can be conducted with the tags in place, and imaging can be imported to observe the new locations and orientations of the tags. The 3D image is deformed according to a volume maintaining algorithm that aligns the imaged tags to the live observed tags. In some embodiments, the volume maintaining algorithm is physics and physiology-based that may incorporate tissue density, elasticity, deformation, etc.

### D. Wireless Tag Deployment

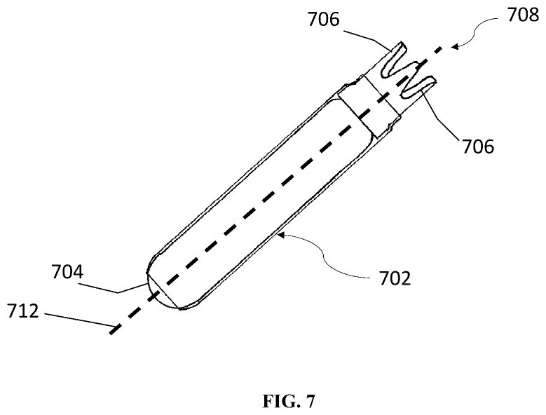

Tags can be delivered into soft tissue through needles, catheters, etc. with a conventional plunger mechanism. In some embodiments, additional mechanical features are added to secure the wireless tag to surrounding tissue. Securing the wireless tag is especially important during tissue excision involving manipulation of the tissue. With reference to FIG. 7, the wireless localization tag 702 is positioned within a housing 704 having at least one tine 706 formed at an axial end 708 of the housing 704. In the illustrated embodiment, the tines 706 extend along a longitudinal axis 712 of the tag 702. The tines 706 are configure to grip, secure, or otherwise anchor the wireless tag 702 to surrounding tissue.

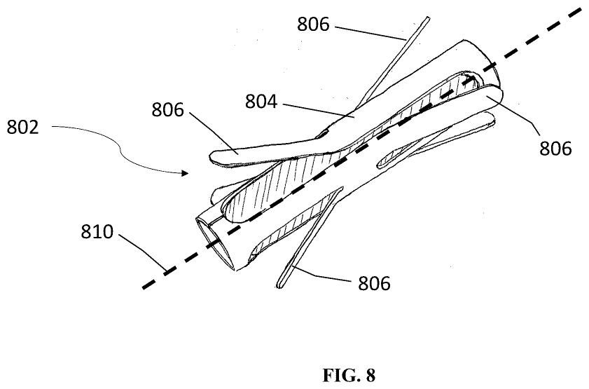

With reference to FIG. 8, a wireless tag 802 includes a housing 804 with self-deploying tines 806 that engage in surrounding tissue and prevent movement. In the illustrated embodiment, the tines 806 deflect radially with respect to a longitudinal axis 810 of the tag 802 (housing 804). In some embodiments, the tines 806 are movable to a first position where the tines 806 are deflected radially inwards toward the longitudinal axis 810 (e.g., during deployment or delivery), and the tine 806 are movable to a second position where the tines 806 extend radially outwards from the longitudinal axis 810 (e.g., deployed and in position). In other words, the tines 806 deploy radially outward when in position. In some embodiments, the tines 806 are used to secure the wireless tag 802 to a patient's lungs.

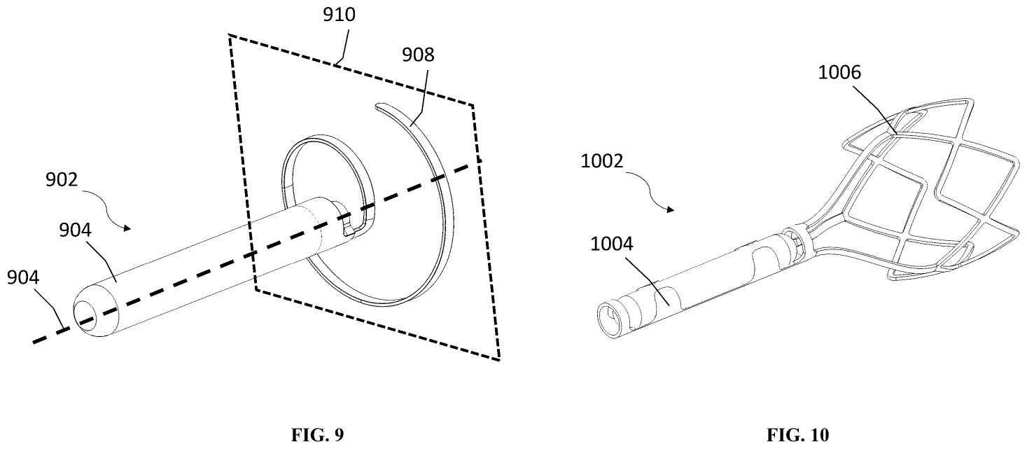

With reference to FIG. 9, a wireless tag 902 includes a housing 904 with a tine 908. In the illustrated embodiment, the tine 908 is a spiral that extends in a plane 910 perpendicular to a longitudinal axis 912 of the tag 902. In some embodiments, the tine 908 is made of nitinol. The tine 908 is movable between a first position where the spiral tine 908 is deflected radially inward toward the longitudinal axis 912 (e.g., a circumferentially compressed deploying position) and a second position where the spiral tine 908 extends radially outward (e.g., a deployed position). In the illustrated embodiment, the spiral tine 908 remains in the plane 910 as the tine 908 moves between the first position and the second position.

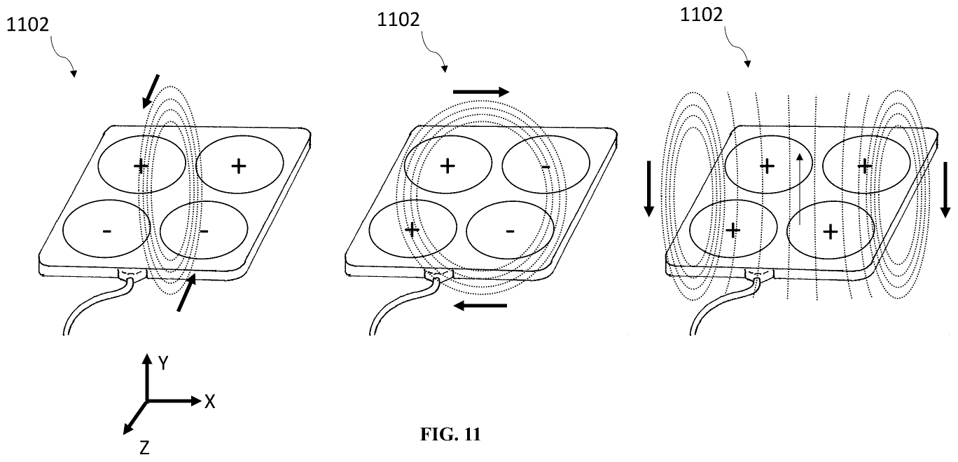

With reference to FIG. 10, a wireless tag 1002 includes a housing 1004 with a tine 1006. In the illustrated embodiment, the tine 1006 is a deployable stent.

### E. Targeted Radiation Delivery

When delivering high energy radiation to soft tissue of a patient, positioning the target tissue at the intended location is critical. Conventional techniques to determine the location to radiate include use of external landmarks (e.g., anatomy, tattoos) or x-ray-based imaging. Conventional techniques to detect patient movement after placement include monitoring an external tag, monitoring patient breaching via air flow.

A solution presented herein provides direct localization with a tag implanted at or near the site targeted for radiation. If a wireless tag is implanted at the site the radiation is intended to be delivered, the patient will necessarily be positioned such that the tag is at or near the focus of the targeted radiation systems. Therefore, the volume over which a wireless tag is placed is relatively small (e.g., 10 cm×10 cm×10 cm) compared to other procedures. The tag excitation and sensing elements should not overlap with the radiation to both prevent beam deflection and also prevent damage to the electronics. In some embodiments, it is advantageous to have the excitation and sensing elements in separate modules, such that the excitation field is much lower and electronic filters are sufficient to reject the excitation signal. In some embodiments, a Helmholtz coil configuration, with exciters at two locations with the wireless tag between the two, may also be advantageous. In other embodiments, a single pad with both exciter and sensors is placed alongside the patient during radiation treatment, instead of under the patient.

### F. Perioperative Imaging

Radiopaque materials used in wireless tag signal excitation and sensing can obscure patient features when present between the imaging equipment source and detectors. In some procedures, a signal is provided when the wire, catheter, or similar device is at a certain location within the vessel relative to the implant.

A solution presented herein is a wireless tag positioned within a stent (e.g., a coronary vessel) when it is desired, for example, to retrieve the stent. A solution also includes a wired emitter is coupled to an outer wall of a catheter or similar device. The wired emitter provides enough magnetic field strength to power up the wireless tag. A sensor or set of sensors position outside the body listen for the wireless tag response (indicating the wired emitter is approaching the wireless tag). In some embodiments, the signal conditioning on the sensor signal includes a notch or low pass filter to block the exciter signal carried by the emitter. If the wireless tag is in range of the wired emitter, the frequency spectrum characteristic of the wireless can be sensed.

Alternatively, the implant includes a magnet, and the catheter includes a high-sensitivity magnetometer. This approach includes a “zeroing” procedure to reduce effect of variation in the local magnetic field due to the earth and nearby metallic components.

Alternatively, the tag includes a passive highly resonant LC circuit, and the reflected signal on the emitter is monitored. Changes in coupling between the wired emitter and implant alters the reflected signal, and a location where the reflected signal is affected the most would correspond to the position of maximum coupling, and the geometry of the emitter and tag are tailored such that the position of maximum coupling corresponds to a signal relative location of the catheter within the vessel.

### G. Arterial Access

Gaining arterial access can be challenging especially for femoral access in either cardio or leg interventions. A solution presented herein is a wireless tag positioned along the artery at the location of intended access under ultrasound or imaging guidance. In some embodiments, the needle used for initial access is tracked with a wireless or wired beacon, then guided by the system for ideal placement and direction. High precision reduces bleeding and reduce the time required to gain access.

### H. Different Environment

Changes in the complex magnetic permeability near the pad can influence the signals used to localize beacons. For example, metallic (high conductivity) materials force the magnetic field to zero by means of induced currents that act as signal source at the same frequency. Ferrous (high permeability) materials are much less common but also modify the fields. Pads positioned under the patient are placed as close as possible to beds with large metallic components. A mapping process is used to learn the influence of the bed on the fields, and remove the influence on localization. However, different environments range from including no metal to mounting a pad directly on metal. Solutions presented herein permit the pad to be used in these various environments.

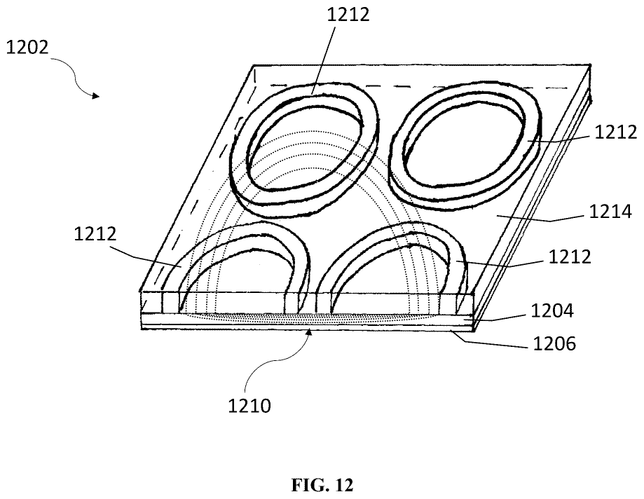

A first challenge arises from large metal loops embedded in bed that can create an opposing magnetic field that effectively cancels the exciter field. A solution presented herein is to position an electrically conductive layer 1206 (e.g., conductive plate) on the bottom of the pad 1202. The conductive plate 1206 acts as a shield to changes in environment permeability below the pad 1202. With reference to FIG. 12, the conductive plate 1206 shapes the magnetic field (see arrow 1210) to contain the electromagnetic flux in the pad 1202. In some embodiments, the conductive plate is metallic. In some embodiments, the conductive layer is aluminum. In some embodiments, the conductive layer is mu-metal, cooper, or stainless steel. In some embodiments, the electrically conductive layer includes a conductivity of at least 20×10⁶ Siemens per meter (S/m).

A second challenge arises from the conductive layer affecting the exciter field because of currents induced in the metal and the resulting opposing magnetic field. With reference to FIG. 12, a solution presented herein is a pad 1202 with a first layer 1204 of a high electromagnetically permeability material and a second layer 1206 of electrically conductive material (e.g., the conductive plate). In some embodiments, the first layer 1204 has an electromagnetic permeability within a range of approximately 10 to approximately 5000. In some embodiments, the first layer 1204 is a ferrite core (e.g., oxides made from Iron, Manganese, and Zinc-manganese zinc ferrites). In some embodiments, the first layer 1204 is a composite including Iron, Silicon, Aluminum, and/or Nickel (e.g., a powder core)

In the illustrated embodiment, the first layer 1204 is positioned between a plurality of exciter coils 1212 and the second layer 1206. The electromagnetic field at second layer 1206 is lower and the corresponding currents induced lower. In the illustrated embodiment, the permeable layer 1202 is positioned between the exciter coil 1212 and the conductive layer 1206. As such, the pad 1202 includes a combination of materials that redirects and contains the magnetic flux on the bottom of the pad 1202, and the pad 1202 is configured to operate efficiently on various surgical beds (e.g., “bed agnostic”).

In some embodiments, the exciter coils 1212 are encapsulated with a high thermal conductivity encapsulating material 1214 (e.g., an epoxy). In some embodiments, the encapsulating material has a thermal conductivity greater than 1000 W/Kelvin. In some embodiments, the encapsulating material has a heat capacity greater than 1000 Joules per kg° C. In some embodiments, the encapsulating material 1214 has a dielectric strength of at least 400 Volts per mil. In some embodiments, ferrite material helps shape the magnetic field.

A third challenge arises because the presence of a high permeability material distorts the magnetic field directions. Distortion of the magnetic field directions creates challenges because it is desirable to position the sensors in a location and orientation where the sensor is orthogonal to the exciter field for each of the states of the exciter creating different field directions. The distortion is pronounced in multi-exciter systems where the phases of the current are varied to change the primary directionality of the induced magnetic field. This effect results in large changes in field direction, in the plane of the exciter, as the exciter is configured to create different field direction (e.g., FIG. 11).

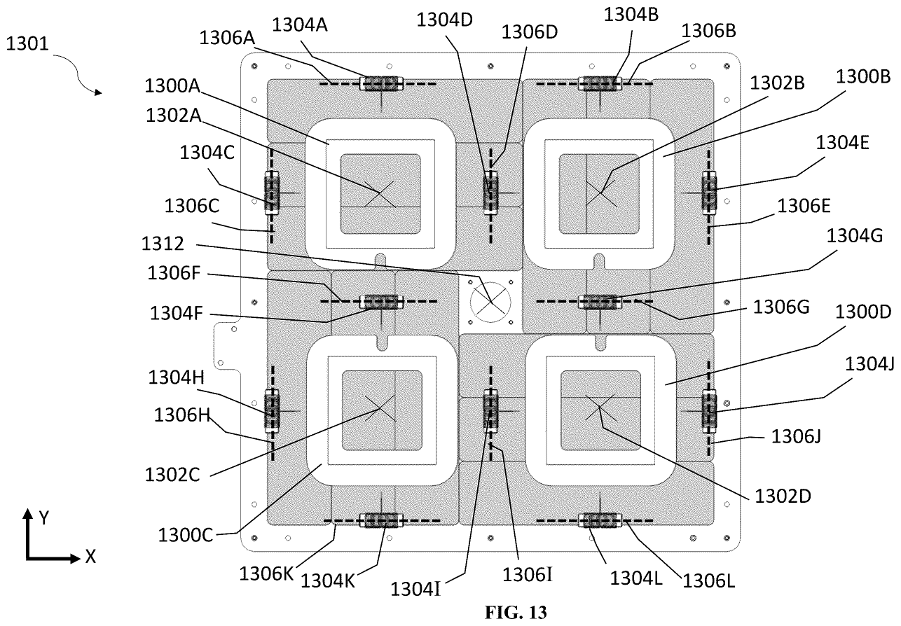

With reference to FIG. 13, a solution presented herein is a pad 1301 with exciter coils 1300A, 1300B, 1300C, 1300D and the sensor coils 1304A-1304L arranged in a grid configuration. The sensor coils 1304A-1304L are placed in locations where the least change in field direction in the plane of the exciter occurs. In the illustrated embodiment, the sensor coils 1304A-1304L each define a sensor coil axis 1306A-1306L and the exciter coils 1300A-1300D each define an exciter coil axis 1302A-1302D. In this grid configuration, all sensors coils 1304A-1304L are oriented parallel to the tangent of adjacent exciter coil 1300A-1300D. For example, the sensor coil axes 1306A, 1306D, 1306F, and 1306C of sensors coils 1304A, 1304D, 1304F, 1304C are oriented parallel to the tangent of the exciter coil 1300A. In other words, the sensor coil axes 1306A, 1306D, 1306F, and 1306C do not intersect the exciter coil axis 1302A.

With continued reference to FIG. 13, the four exciter coils 1300A-1300D are positioned circumferentially around a center 1312 of the pad. In the illustrated embodiment, the sensors axes 1306A, 1306B, 1306F, 1306G, 1306K, and 1306L are aligned in a first direction (e.g., an X direction) and the sensor axes 1306C, 1306D, 1306E, 1306H, 1306I, and 1306J are aligned in a second direction (e.g., a Y direction). In the illustrated embodiment, the first direction and the second direction are perpendicular. In the illustrated embodiment, the excitor axes 1302A-1302D are aligned in a third direction perpendicular to the plane of view of FIG. 13 (e.g., a Z direction). In the illustrated embodiment, the sensor coils 1304A-1304L are positioned centered with at least one exciter coil (in line with at least one exciter axis 1302A-1302D). For example, the sensor coils 1304A, 1304D, 1304F, and 1304C are positioned circumferentially around the exciter coil 1300A. In the illustrated embodiment, there is a sensor coil 1304A- 1304L positioned at the mid-point of each side of each exciter coil 1300A-1300D.

In some embodiments, the exciter coil is circular. In other embodiments, the exciter coil is rectangular or another suitable shape. In some embodiment, the electronics utilized for monitoring and controls of the pad are positioned under the conductive shield (e.g., the conductive shield is positioned between the electronics and the exciter coils).

In the illustrated embodiment, the high permeability material tolerates high fields without becoming saturated. In some embodiments, the internal induced field the high permeability material can support is higher than the external applied field.

Various features and advantages are set forth in the following claims.

What is claimed is:

1. A wireless localization system comprising:
a pad including an exciter coil and a sensor coil;
a first wireless tag configured to generate a first signal in response to a magnetic field generated by the exciter coil, wherein the first signal is detected by the sensor coil;
a second wireless tag coupled to a tissue of a patient, the second wireless tag configured to generate a second signal in response to the magnetic field generated by the exciter coil, wherein the second signal is detected by the sensor coil;
a processor configured to determine a location of the first wireless tag and the second wireless tag based on the first signal and the second signal detected by the sensor coil; and
wherein a desired perspective is determined by the processor in response to the first wireless tag moving to a plurality of registration positions; and
a control console, and wherein the location of the second wireless tag is displayed on the control console from the desired perspective.

2. The system of claim 1, further comprising a camera with a field of view from a camera perspective; and wherein the desired perspective is the camera perspective.

3. The system of claim 2, wherein the location of the second wireless tag is displayed on the control console overlayed with the field of view from the camera.

4. The system of claim 2, wherein the plurality of registration positions includes an upper-right corner of the field of view, a lower-right of the field of view, an upper-left corner of the field of view, and a lower-left corner of the field of view.

5. The system of claim 2, wherein the plurality of registration positions includes at least one position at a first distance from the camera and at least one position at a second distance from the camera.

6. The system of claim 2, wherein the first wireless tag is positioned on a surgical tool.

7. The system of claim 2, wherein the first wireless tag is positioned on a surgical robotic arm.

8. The system of claim 2, wherein the first wireless tag is positioned in a moveable object with an outer shell, an inner sphere movable with respect to the outer shell, wherein the fourth wireless tag is positioned within the inner sphere.

9. The system of claim 8, wherein the inner sphere includes a weighted portion to orient the sphere in a default orientation with respect to gravity.

10. The system of claim 2, further comprising a third wireless tag positioned on the camera; wherein the third wireless tag is configured to generate a third signal in response to the magnetic field generated by the exciter coil, wherein the third signal is detected by the sensor coil, and wherein the processor is configured to determine a location of the third wireless tag based on the third signal detected by the sensor coil.

11. The system of claim 1, wherein the tissue that the second wireless tag is coupled to is one of a lung tissue, a bone tissue, a soft tissue, and an artery.

12. The system of claim 1, wherein the second wireless tag comprises an outer housing including an anchor, wherein the anchor is configured to be secured within the tissue of the patient; and wherein the anchor extends radially outward from a longitudinal axis of the outer housing.

13. The system of claim 1, wherein the exciter coil is a first exciter coil and the pad further includes a second exciter coil, a third exciter coil, and a fourth exciter coil positioned circumferentially around a center; and wherein the magnetic field generated by the first exciter coil, the second exciter coil, the third exciter coil, and the fourth exciter coil comprises three orthogonal magnetic fields.

14. The system of claim 13, wherein the sensor coil is a first sensor coil, the pad further including a second sensor coil, a third sensor coil, and a fourth sensor coil; wherein the first sensor coil, the second sensor coil, the third sensor coil, and the fourth sensor coil are positioned circumferentially around the first exciter coil.

15. The system of claim 14, wherein the first sensor coil includes a first sensor axis and the third sensor coil includes a third sensor axis, wherein the first sensor axis is parallel to the third sensor axis, and
wherein the second sensor coil includes a second sensor axis and the fourth sensor coil includes a fourth sensor axis, wherein the second sensor axis is parallel to the fourth sensor axis; and
wherein the first sensor axis is perpendicular to the second sensor axis; and wherein the first exciter coil includes an exciter coil axis perpendicular to the first sensor axis and the second sensor axis.

16. The system of claim 15, further comprising a camera with a field of view from a camera perspective; and wherein the desired perspective is the camera perspective.
